(19) United States
(12) Patent Application Publication
(10) Pub. No.: US 2005/0014965 A1
(43) Pub. Date: Jan. 20, 2005

(54) Method of separating metallic catalyst constituents from reaction mixtures

(76) Inventors: Marc Dahlmann, Dusseldorf (DE); Peter Fischer, Koln (DE)

Correspondence Address: BAYER MATERIAL SCIENCE LLC, 100 BAYER ROAD, PITTSBURGH, PA, 15205, US

(21) Appl. No.: 10/891,678

(22) Filed: Jul. 15, 2004

(30) Foreign Application Priority Data
Jul. 18, 2003 (DE) 10332697.9

(51) Int. Cl. C07C 69/96

(52) U.S. Cl. 558/270

(57) ABSTRACT

A process for the preparation of an aromatic carbonate is disclosed. The process entails reacting in the presence of a catalyst system an aromatic hydroxy compound with carbon monoxide and oxygen, and optionally in one or more solvents to produce a liquid phase. At least a portion of the liquid phase is subjected to a treatment to obtain a treated liquid phase. The treatment entails at least one of (a) heating to a temperature that is at most mean reaction temperature without passing oxygen thereto, and (b) adding one or more protic compounds thereto, and (c) passing through it one or more inert or reducing gases. Solid metallic catalyst constituents are then separated from the treated liquid phase.

### FIELD OF THE INVENTION

The invention relates to a separation process and in particular to the separation of catalyst constituents from a liquid phase.

### SUMMARY OF THE INVENTION

A process for the preparation of an aromatic carbonate is disclosed. The process entails reacting in the presence of a catalyst system an aromatic hydroxy compound with carbon monoxide and oxygen, and optionally in one or more solvents to produce a liquid phase. At least a portion of the liquid phase is subjected to a treatment to obtain a treated liquid phase. The treatment entails at least one of (a) heating to a temperature that is at most mean reaction temperature without passing oxygen thereto, and (b) adding one or more protic compounds thereto, and (c) passing through it one or more inert or reducing gases. Solid metallic catalyst constituents are then separated from the treated liquid phase.

### BACKGROUND OF THE INVENTION

The preparation of diaryl carbonates (DAC) by the oxidative carbonylation of aromatic hydroxy compounds by means of carbon monoxide and oxygen is known. The reaction is mediated by a catalyst system containing a noble metal. Palladium is preferably used as the noble metal. There may additionally be used a co-catalyst (e.g. manganese, copper, lead, titanium or cobalt salts), a base, bromide sources, quaternary salts, various quinones or hydroquinones and drying agents. It is possible for the operation to be carried out in a solvent.

The reaction mixtures formed by contact of the aromatic hydroxy compound with carbon monoxide and oxygen in the presence of the catalyst system contain, in addition to the diaryl carbonate, unreacted phenol and, optionally, a solvent, constituents of the catalyst system, which generally comprises several components (hereinafter referred to as DAC-Forming Reaction Mixture or Reaction Mixture, the term Reaction as used below refers to the DAC-Forming Reaction and the term Liquid Phase as used below refers to the liquid phase resulting upon the Reaction). For the described process to be carried out economically, it is necessary to separate the individual components, especially the noble metal component, from the product stream and, optionally after a regeneration step, return these components to the reaction.

Only a small number of methods of separating metallic catalyst constituents from Liquid Phase are known.

EP-A 0 913 197 describes the removal of catalyst components by extraction of the product stream using aqueous solutions. Palladium may be precipitated from the aqueous extract by addition of a reducing agent.

Alternatively, as taught by EP-A 1 140 775, the precipitation of palladium from the aqueous extract may be effected by addition of a precipitating agent, such as, for example, salts of oxalic acid or of acetylacetone.

Both methods are associated with considerable outlay in terms of apparatus. For example, for the aqueous extraction of the metal-containing catalyst constituents from the reaction mixture, at least one extraction column or a mixer/separator combination is required. Isolation of the metal-containing catalyst constituents from the aqueous extract additionally requires the addition of a reagent which is capable of converting the dissolved metal compounds into an insoluble form or which reacts with the dissolved metal compounds to form a sparingly soluble compound. Further reaction apparatuses are necessary for these reactions. In addition, the required reagents frequently give rise to considerable costs, which have an adverse effect on the economy of the DAC preparation process as a whole. Furthermore, the required reagents are frequently foreign substances which are not used in the Reaction. Accordingly, when the metallic catalyst components that have been separated off are returned to the Reaction, contamination with the foreign substances used for isolating the metallic catalyst components is possible. This in turn may have an adverse effect on the reactivity and selectivity of the catalyst used.

Accordingly, the object of the present invention is to provide a simple process for separating one or more metallic catalyst components from the product stream from the preparation of aromatic carbonates by oxidative carbonylation of hydroxy aromatic compounds, which process does not have the disadvantages mentioned above.

### DETAILED DESCRIPTION OF THE INVENTION

It has now been found, surprisingly, that the content of metallic catalyst constituents may be significantly reduced or completely removed from the Liquid Phase by a process that entails treatment of the Liquid Phase followed by its filtration or by other solid/liquid separation operation. The inventive method is simple and in particular makes only small demands in terms of apparatus. Furthermore, it is possible to dispense with the use of expensive reagents or of reagents that are not normally used in the reaction system of the DAC preparation.

The inventive process relates to the preparation of aromatic carbonate of formula (I)
Ar—O—CO—O—Ar (I)
wherein Ar is an aromatic organic radical, preferably a phenyl radical. Accordingly an aromatic hydroxy compound of formula (II)
Ar—O—H (II),
is reacted in a liquid phase with carbon monoxide and oxygen, optionally in a solvent and in the presence of a catalyst system. The catalyst system includes one or more members selected from the first group consisting of the compounds of Ru, Os, Rh, Ir, Pd and of Pt, and one or more members selected from the second group consisting of the compounds of Al, Ga, In, Ti, Sc, Y, La, Ge, Sn, Pb, Ti, Zr, Hf, V, Nb, Ta, Cu, Ag, Au, Zn, Cd, Hg, Cr, Mo, W, Mn, Tc, Re, Fe, Co, Ni and of an element having an atomic number from 58 to 71 (a rare earth metal). The reaction is carried out at a mean reaction temperature T of from 60 to 140° C.

The inventive process entails in sequence obtaining the Liquid Phase, treatment of the Liquid Phase and separating the metallic catalyst constituents in solid form from the Liquid Phase.

The treatment comprises at least one of steps (a), (b) and (c), where
(a) refers to heating of the Liquid Phase to a temperature that is 0 to 80° C. below T for a period of 30 seconds to 10 hours without passing oxygen into the Liquid Phase, and
(b) refers to adding to the Liquid Phase one or more protic compounds, and
(c) refers to passing through the Liquid Phase one or more gases which, under the prevailing conditions, are either inert or have a reducing action for the metallic catalyst constituents.

The DAC-Forming Reaction Mixtures on which the process according to the invention may be used are preferably the ones resulting from the oxidative carbonylation of aromatic hydroxy compounds Ar—O—H (II), such as, for example, monohydroxy compounds, such as phenol, o-, m- or p-cresol, o-, m- or p-chlorophenol, o-, m- or p-ethylphenol, o-, m- or p-propylphenol, o-, m- or p-methoxyphenol, 2,6-dimethylphenol, 2,4-dimethylphenol, 3,4-dimethylphenol, 1-naphthol, 2-naphthol, or di- or poly-hydroxy compounds, such as resorcinol and hydroquinone, as well as tris- and bis-phenols, such as 2,2-bis-(4-hydroxyphenyl)-propane (bisphenol A), 2,2-bis-(3,5-dimethyl-4-hydroxyphenyl)-propane, 1,1-bis-(4-hydroxyphenyl)-cyclohexane, 1,1-bis-(4-hydroxyphenyl)-3,3,5-trimethyl-cyclohexane or 6,6′-dihydroxy-3,3,3′,3′-tetramethyl-1,1′-spiro(bis)-indane, 2,4′-hydroxybiphenyl or 4,4′-hydroxybiphenyl.

If the aromatic hydroxy compound is substituted, it is generally substituted by from 1 to 3 substituents such as C₁-C₁₈-alkyl, C₆-C₁₈-aryl, C₇-C₁₈-aralkyl, C₁-C₁₈-alkoxy, fluorine, chlorine or bromine.

The term DAC-Forming Reaction Mixture as used in the present context applies also to reaction mixtures resulting from the oxidative carbonylation of monohydroxy compounds, particularly preferably on reaction mixtures from the oxidative carbonylation of phenol.

The Reaction Mixtures preferably contain platinum metal catalysts (III), preferably those which contain at least one noble metal of group VIII, especially palladium. The catalysts, especially palladium, may be used in various forms in the Reaction. Palladium may be used, for example, in metallic form, for example in the form of palladium black or, preferably, in the form of palladium compounds of oxidation states 0 and +2, such as, for example, palladium(II) acetylacetonate, halides, carboxylates of C₂-C₁₈-carboxylic acids, dicarboxylates such as oxalate, nitrate, sulfate, oxides or palladium complexes, which may contain, for example, carbon monoxide, olefins, amines, nitrites, phosphorus compounds and halides. Particular preference is given to the use of palladium bromide and palladium acetylacetonate.

The amount of platinum metal catalyst in the Reaction is not limited. The amount of catalyst used is preferably such that the concentration of the metal in the Reaction Mixture is from 1 to 3000 ppm, concentrations of from 5 to 500 ppm being particularly preferred.

A second metal salt, which acts as co-catalyst, in the Reaction Mixture is at least one salt of a metal selected from among groups III A, III B, IV A, IV B, V B, I B, II B, VI B, VII B, the rare earth metals (atomic numbers 58-71) or the iron group of the periodic system of the elements (Mendeleyev), optionally also mixtures thereof, it being possible for the metal to be used in various oxidation states.

U.S. Pat. No. 5,142,086, U.S. Pat. No. 5,231,210, U.S. Pat. No. 5,284,964, EP-A 0 350 697, EP-A 0 350 700 and U.S. Pat. No. 5,336,803, all incorporated herein by reference disclose such compounds.

Preference is given to the use of Pb, Ti, Mn, Cu, Co, V, Zn, Ce and Mo. Without limiting the process according to the invention there may be mentioned lead(II), cerium(III), manganese(II), manganese(III), copper(I), copper(II), cobalt(II), cobalt(III), vanadium(III) and vanadium(IV). The metals may be used, for example, in the form of halides, oxides, carboxylates of C₂-C₁₈-carboxylic acids, diketonates or nitrates and also in the form of complex compounds, which may contain, for example, carbon monoxide, olefins, aromatic and aliphatic mono- or poly-amines, phosphorus compounds, pyridines, bipyridines, terpyridines, quinolines, isoquinolines, cryptands, Schiffs bases and halides.

Particular preference is given to the use of Mn, Cu, Mo, Ti, Pb and Ce. Very particular preference is given to the use of manganese compounds, particularly preferably manganese(II) and manganese(III) complexes, very particularly preferably manganese(II) acetylacetonate and manganese(III) acetylacetonate, as well as manganese(II) bromide.

The co-catalyst, which may also be formed in situ, is used in an amount such that its concentration is preferably in the range of from 0.0001 to 20 wt. % of the Reaction Mixture; preference is given to the concentration range from 0.001 to 5 wt. %, particularly preferably from 0.005 to 2 wt. %.

There are used as optional components, for example, bromide compounds, bases or solvents.

The bromide compounds optionally present in the Reaction Mixture include alkali bromides or alkaline earth bromides, preferably bromide salts of organic cations.

Suitable organic cations include ammonium, guanidinium, phosphonium or sulfonium salts substituted by organic radicals, optionally also mixtures thereof. Particularly suitable for use in the process according to the invention are ammonium, guanidinium, phosphonium and sulfonium salts which contain as organic radicals C₆- to C₁₀-aryl, C₇- to C₁₂-aralkyl and/or C₁- to C₂₀-alkyl radicals.

In the process according to the invention there are preferably used ammonium salts which carry as organic radicals C₆- to C₁₀-aryl, C₇- to C₁₂-aralkyl and/or C₁- to C₂₀-alkyl radicals; tetrabutylammonium bromide and tetrabutylphosphonium bromide are particularly preferred.

The amount of such a quaternary salt may be, for example, from 0.1 to 20 wt. %, based on the weight of the Reaction Mixture. This amount is preferably from 0.5 to 15 wt. %, particularly preferably from 1 to 5 wt. %.

Examples of bases which may be employed in the Reaction include alkali hydroxides, alkali salts or quaternary salts of weak acids, such as alkali tert.-butoxides, or alkali salts or quaternary salts of aromatic hydroxy compounds of formula (II), in which Ar is as defined. Very particular preference is given to the use of an alkali salt or quaternary salt of the aromatic hydroxy compound of formula (II) that is also to be reacted to the organic carbonate, for example tetrabutylammonium phenolate or potassium phenolate.

The alkali salts may be lithium, sodium, potassium, rubidium or caesium salts. Preference is given to the use of lithium, sodium and potassium phenolates, particularly preferably potassium phenolate.

The quaternary salts may be ammonium, phosphonium, pyridinium, sulfonium or guanidinium salts which possess as organic radicals C₆- to C₁₈-aryl, C₇- to C₁₈-aralkyl and/or C₁- to C₂₀-alkyl radicals. The radicals may all be identical or different, mixtures of several quaternary salts may optionally be used. It is preferred, where appropriate, to use the same cation that is also used as bromide for the above-mentioned bromide compound. Also preferred are tetraphenylphosphonium, tetrabutylammonium, tetrabutylphosphonium; tetrabutylammonium is particularly preferred.

Alternatively, it is also possible to use trialkylamine bases, such as tributylamine, diisopropylethylamine, DBU, DBN.

The base is preferably added in an amount that is independent of the stoichiometry. The ratio of platinum metal, e.g. palladium, to base is preferably so chosen that from 0.1 to 5000, preferably from 1 to 1000, particularly preferably from 10 to 300 equivalents of base are used per mole of platinum metal.

Solvents that are inert under the reaction conditions may optionally be used and be present in the DAC-Forming Reaction Mixture. Examples of solvents which may be mentioned include aliphatic hydrocarbons, such as pentane, petroleum ether, cyclohexane, isooctane, aromatic hydrocarbons, such as benzene, toluene, xylenes, chloroaromatic compounds, such as chlorobenzene or dichlorobenzene, ethers, such as dioxane, tetrahydrofuran, tert.-butyl methyl ether, anisole, amides, such as dimethylacetamide, N-methyl-pyrrolidinone, alcohols, such as tert.-butanol, cumyl alcohol, isoamyl alcohol, diethylene glycol, tetramethylurea.

Mixtures of solvents may be used. The inert solvent may be present in the reaction mixture in an amount of from 1 to 99%, preferably from 20 to 98%, particularly preferably from 30 to 98%. Especially when using bromide compounds or bases it is advantageous to employ solvents which impart solubility to inorganic salts such as NaBr or NaOPh, such as, for example, dipolar aprotic solvents (e.g. N,N-dimethylformamide, N,N-dimethylacetamide, N-methylpyrrolidinone, sulfolane, acetonitrile) or crown ethers, cryptands or “open crown ethers” (e.g. diethylene glycol dimethyl ether, triethylene glycol dimethyl ether, tetraethylene glycol dimethyl ether).

The inventive process is performed on the Liquid Phase immediately after completion of the Reaction. The inventive process may be carried out on approximately from 1 to 100 wt. % of the total amount of the Liquid Phase.

This amount is dependent on the particulars of the process. For example, if a portion of the Liquid Phase is recycled to the Reaction, the inventive process (herein Treatment) may be carried out on that portion that is not recycled, and the recycled portion undergoes no Treatment. It is also possible for the Treatment to be applied only to the portion of the Liquid Phase that is recycled to the Reaction. Preferably 70 to 100 wt. %, particularly preferably 90 to 100 wt. %, of the Liquid Phase that is not recycled directly to the Reaction is subjected to the Treatment.

The period of time for which the thermal Treatment (see embodiment (a) below) is carried out is approximately from 30 seconds to 10 hours and covers an amount of less than 50%, preferably less than 35%, of the total time or mean dwell time of the Reaction that precedes it.

In one particular embodiment (a) of the present invention, the Liquid Phase is subjected to temperatures which are on average from 0 to 100° C., preferably from 0 to 80° C., particularly preferably from 0 to 15° C., below the mean temperature of the Reaction.

Within the scope of the invention, the mean thermal Treatment temperature is understood to be the quotient of the integral of the plotting of the Treatment temperature against the Treatment time (or dwell time) and of the Treatment time (or dwell time).

Within the scope of the invention, the mean reaction temperature is understood to be the quotient of the integral of the plotting of the Reaction temperature against the Reaction time (or dwell time) and of the Reaction time (or dwell time).

The mean Treatment temperature is approximately in the range of from 30 to 120° C.

While the temperature profile of the thermal Treatment of embodiment (a) is not critical to the invention, the use of a constant temperature or of temperature-time profile with a monotonically negative gradient is preferred. It is particularly preferred for the starting temperature of the Treatment to differ from the final temperature of the Reaction by less than 5° C.

Because the inventive Treatment normally follows directly the Reaction, the starting temperature of the Treatment is typically also the final temperature of the Reaction.

It is a preferred characteristic of the heat Treatment that no oxygen or other oxidizing compound is passed into the Liquid Phase during this time. Even if introduced gases have a residual oxygen content, less than 10 standard liters per hour and per liter of reaction apparatus volume are passed into the Liquid Phase.

In a further embodiment, (c) of the Treatment gas is passed through the Liquid Phase.

Suitable gases include inert gases, such as nitrogen and carbon dioxide, or noble gases, such as helium, neon and argon. Other preferred gases include gases having reducing properties. Within the scope of the invention, reducing properties are understood to mean the ability, under the reaction conditions, to give up electrons to the metallic catalyst constituents in oxidized form. Examples of gases having reducing properties are carbon monoxide and hydrogen. The use of carbon monoxide is particularly preferred.

The pressure is approximately from 0.01 to 500 bar; the pressure used is preferably less than or equal to the mean pressure of the Reaction.

A further particular embodiment (b) of the Treatment entails adding one or more protic compounds during the after-treatment of the reaction mixture. Examples of protic compounds include mono- or polyhydric aliphatic or aromatic alcohols, mono- or polyvalent aliphatic or aromatic carboxylic acids, aliphatic and aromatic amines and water, as well as dilute inorganic acids and salt solutions. The use of water or a dilute aqueous solution is particularly preferred. Because water is also formed as a reaction product, it is optionally possible to dispense with the removal of water from the reaction mixture towards the end of the reaction and hence achieve a marked increase in the water content of the reaction mixture.

The protic compound is added in a positive amount less than 0.1 part by volume relative to the total volume of the Liquid Phase. Preferably less than 0.05 part by volume, particularly preferably less than 0.01 part by volume, is added. If the added protic compound has only limited miscibility with the reaction mixture, the maximum amount of the protic compound added to the reaction mixture is the amount which is sufficient to reach the miscibility gap of the mixture comprising the reaction mixture and the protic compound

Embodiments (b) and (c) may be combined by passing the vapor of a protic compound, for example water vapor or superheated methanol, through the Liquid Phase.

Separation of the metallic catalyst constituent following the Treatment is preferably carried out by a method of solid/liquid separation. Possible methods include, for example, techniques of vacuum, pressure or centrifugal filtration, sedimentation and sedimentation centrifugation. A combination of different techniques of solid/liquid separation is likewise possible.

The preferred method of solid/liquid separation is sedimentation centrifugation. Sedimentation centrifuges having a large equivalent clarifying surface, such as disk separators, are preferred. Particular preference is given to the use of self-desludging disk separators, very particularly preferably disk separators with discharge via discharge ploughs, for example type SB 150 from Westfalia Separator AG.

The thermal Treatment may be carried out in the presence of a solid that is insoluble in the Liquid Phase. A porous solid is preferably used for that purpose. Examples of solids which may be used include kieselguhr, perlite, glass powder, cellulose fibers, talc and porous plastics particles, as well as substances which are also used as supports for heterogeneous catalysts, such as metal oxides from the group V, Mn, Ti, Cu, Zr, La, the rare earth metals (atomic numbers 58-71), both as chemically uniform pure substances and in a mixture, as well as iron and cobalt oxides, nickel, aluminium, silicon and magnesium oxide, zeolites and activated carbons.

The addition of the solid may be carried out either at the beginning of the thermal Treatment or during or after the thermal treatment of the Liquid Phase. The addition of the solid is preferably carried out before the last solid/liquid separating operation in the process according to the invention.

The process according to the invention may be preceded or followed by further working-up steps for separating off the same or other catalyst components, solvents, starting material or products.

For example, distillations for separating off solvent and/or phenol and/or for separating off some DPC at excess, normal and reduced pressure may take place between the Reaction and the Treatment according to the invention. A preferred embodiment of the thermal Treatment (a) according to the invention entails simultaneously separating off volatile components by distillation. This process may also be carried out as a stripping process with the passing through of gases or vapors, i.e. may be combined with embodiment (c) according to the invention.

Extractions may be used, for example, for separating off bases, alkali halides, metallic catalyst components or quaternary halides. In the case of a preceding extraction using aqueous solutions, the equilibrium moisture established thereby may be equivalent to the Treatment according to embodiment (b) the addition of a protic compound. The same is true of extractions using other protic compounds.

The process according to the invention may be carried out either continuously or discontinuously.

In the case of a discontinuous procedure, the Treatment may be carried out in a container, into which the Reaction Mixture is transferred after the completion of the DAC-Forming Reaction, or in the reactor for the Reaction.

In the case of a continuous procedure, the Liquid Phase is preferably passed through one or more apparatuses (for example a stirred vessel, a bubble column or a combination of one or more nozzles and a tubular section) which is/are of such a size that the mean dwell time is less than 50% of the dwell time of the actual reactor used for the Reaction.

Suitable reactors for the process according to the invention are stirred vessels, tubular reactors and bubble columns, it being possible for these to be used as individual reactors or as a cascade.

The metallic catalyst constituents separated off by the Treatment may be fed back to the DAC-Forming Reaction either directly or after working up. Working-up steps comprise, for example, reoxidation processes, conversion to halides, carboxylates, acetylacetonates or metal-ligand complexes, which may be used in the reaction again with or without being isolated and worked up. A possible working-up step which may be mentioned by way of example is an oxidative reactivation according to the disclosure of EP-A 0 806 243.

### EXAMPLES

In a continuously operated synthesis apparatus, phenol was reacted with a gas mixture of carbon monoxide and oxygen to form diphenyl carbonate (DPC). Chlorobenzene (MCB) was used as solvent, the catalyst system used consisted of the components palladium(II) bromide, manganese(III) tris(acetylacetonate), tetrabutylammonium bromide and tetrabutylammonium phenolate.

The reaction mixture produced in the synthesis apparatus was immediately treated further as described in the individual examples.

The composition of the reaction mixtures was determined by gas chromatography. The content of MCB, phenol and DPC was determined directly from the gas chromatograms against an internal standard. The content of tetrabutylammonium bromide was calculated from the signal of butyl bromide in the gas chromatogram. The content of tetrabutylammonium phenolate was calculated from the signal of tributylamine in the gas chromatogram, taking into consideration the approximate content of tetrabutylammonium bromide.

The concentrations of the metals were determined by ICP mass spectrometry.

The samples for the metal determination were removed with thorough mixing, so that the measured metal concentrations represented the sum of the concentrations of dissolved and undissolved metal constituents. The samples which were removed were homogenized by digestion before the metal determination.

### Example 1

### All Data in wt. %

A freshly prepared reaction mixture having an approximate composition of 71.3% MCB, 6.6% phenol, 2.2% tetrabutylammonium bromide, 2.0% tetrabutyl-ammonium phenolate and 12.7% diphenyl carbonate and having a palladium content of 14 ppm was placed in a glass reactor. The mixture was adjusted to a temperature of 90° C. and stirred for 60 minutes at that temperature with thorough mixing. The mixture was then passed through a commercial stainless steel deep-bed filter (Pall) having a pore size of 5 μm. The palladium content of the filtrate was 2 ppm (Pd separation: 85% of theory).

### Comparison Example

A freshly prepared reaction mixture having an approximate composition of 71.8% MCB, 7.0% phenol, 2.2% tetrabutylammonium bromide, 2.1% tetrabutyl-ammonium phenolate and 12.6% diphenyl carbonate and having a palladium content of 23 ppm was passed, without further treatment, through a stainless steel deep-bed filter having a pore size of 5 μm. The palladium content of the filtrate was 11 ppm (Pd separation: 52% of theory).

### Examples 2 and 3

A freshly prepared reaction mixture having an approximate composition of 73.9% MCB, 6.6% phenol, 1.9% tetrabutylammonium bromide, 2.5% tetrabutyl-ammonium phenolate and 12.9% diphenyl carbonate was placed in a glass reactor. At a temperature of 90° C., carbon monoxide was passed through the mixture for a period of one hour, with thorough mixing. The mixture was then passed through a stainless steel deep-bed filter having a pore size of 5 μm. The results are shown in Table 1.

TABLE 1

| | Amount of CO gas | Pd content of the | |
|---|---|---|---|
| | (in liters per liter of | starting mixture (in | Pd content after |
| Example | liquid phase) | ppm) | filtration (in ppm) |
| 2 | 10 | 68 | 2 |
| 3 | 50 | 18 | 2 |

### Examples 4 and 5

A freshly prepared reaction mixture having an approximate composition of 73.9% MCB, 6.6% phenol, 1.9% tetrabutylammonium bromide, 2.5% tetrabutyl-ammonium phenolate and 12.9% diphenyl carbonate was placed in a glass reactor, and 0.01 part by volume of water was added thereto. At a temperature of 70° C., carbon monoxide was passed through the mixture for a period of 30 minutes, with thorough mixing. The mixture was then passed through a stainless steel deep-bed filter having a pore size of 5 μm. The results are shown in Table 2.

TABLE 2

| | Amount of CO gas | Pd content of the | |
|---|---|---|---|
| | (in liters per liter of | starting mixture (in | Pd content after |
| Example | liquid phase) | ppm) | filtration (in ppm) |
| 4 | 5 | 21 | 3 |
| 5 | 25 | 57 | 2 |

### Example 6

A freshly prepared reaction mixture having an approximate composition of 71.9% MCB, 6.5% phenol, 2.2% tetrabutylammonium bromide, 2.5% tetrabutyl-ammonium phenolate and 12.6% diphenyl carbonate and having a palladium content of 47 ppm and a manganese content of 210 ppm was placed in a glass reactor. At a temperature of 90° C., 50 standard liters (s.l.) of carbon monoxide per liter of liquid phase are passed through the mixture for a period of one hour, with thorough mixing. A sample of the mixture is then centrifuged in a centrifuge at 6000 rpm. The palladium content of the supernatant was 19 ppm after 2 minutes and 9 ppm after 4 minutes. The manganese content of the supernatant was 220 ppm after 2 minutes and 230 ppm after 4 minutes.

### Example 7

A freshly prepared reaction mixture having an approximate composition of 71.4% MCB, 6.5% phenol, 2.1% tetrabutylammonium bromide, 2.6% tetrabutyl-ammonium phenolate and 12.9% diphenyl carbonate and having a palladium content of 47 ppm and a manganese content of 210 ppm was placed in a glass reactor, and 0.01 part by volume of water was added thereto. At a temperature of 70° C., 25 s.l. of carbon monoxide per liter of liquid phase are passed through the mixture for a period of 30 minutes, with thorough mixing. A sample of the mixture was then centrifuged for 4 minutes in a centrifuge at 6000 rpm. The palladium content of the supernatant was less than 1 ppm. The manganese content of the supernatant was 9 ppm. The solid which was centrifuged off was greasy and could readily be rinsed away.

Example 1 shows that an effective depletion of the palladium content of the reaction mixture may be achieved by thermal treatment of the reaction mixture and subsequent filtration. The comparison example shows that, if the reaction mixture is filtered without previously being subjected to thermal treatment, a markedly higher proportion of the palladium remains in the reaction mixture. The effectiveness of the palladium separation may therefore be markedly increased by thermal treatment of the reaction mixture prior to filtration.

Examples 2 and 3 show that the effectiveness of the thermal treatment of the reaction mixture for the palladium separation by filtration may be increased by passing carbon monoxide into the reaction mixture during the thermal treatment.

Examples 4 and 5 show that, by adding water during the thermal treatment of the reaction mixture, while passing carbon monoxide into the mixture, the effectiveness of the palladium separation during the subsequent filtration may likewise be increased.

Example 6 shows that the palladium may likewise be effectively separated from the reaction mixture after the thermal treatment, while passing carbon monoxide into the mixture, by centrifugation.

Example 7 shows that, by the addition of water during the thermal treatment of the reaction mixture, while passing carbon monoxide into the mixture and with subsequent centrifugation, not only the palladium but also the manganese may be separated from the reaction mixture very effectively.

Although the invention has been described in detail in the foregoing for the purpose of illustration, it is to be understood that such detail is solely for that purpose and that variations may be made therein by those skilled in the art without departing from the spirit and scope of the invention except as it may be limited by the claims.

1. A process for the preparation of an aromatic carbonate of formula I
Ar—O—CO—O—Ar (I)
comprising
reacting in the presence of a catalyst system an aromatic hydroxy compound of formula II
Ar—O—H (II),
wherein
Ar is an aromatic organic radical
with carbon monoxide and oxygen, and
optionally in one or more solvents,
wherein the catalyst system contains at least one member selected from the first group consisting of the compounds of Ru, Os, Rh, Ir, Pd and of Pt,
and at least one member selected from the second group consisting of the compounds of Al, Ga, In, Tl, Sc, Y, La, Ge, Sn, Pb, Ti, Zr, Hf, V, Nb, Ta, Cu, Ag, Au, Zn, Cd, Hg, Cr, Mo, W, Mn, Tc, Re, Fe, Co, Ni and of an element having an atomic number from 58 to 71 (a rare earth metal),
in presence of a liquid phase,
at a mean temperature T of from 60 to 140° C., to obtain a liquid phase that contains solid metallic catalyst constituents
and subjecting at least a portion of the liquid phase to a treatment to obtain a treated liquid phase, the treatment including at least one of (a), (b) and (c) to obtain a treated phase wherein
said (a) denotes heating to a temperature that is 0 to 80° C. below T for a period of from 30 seconds to 10 hours without passing oxygen thererethrough, and
said (b) denotes adding one or more protic compounds thereto, and
said (c) denotes passing therethrough one or more gases which, under the prevailing conditions, are either inert or have a reducing action on the metallic catalyst constituents and separating the solid metallic catalyst constituents from the treated liquid phase.

2. The process according to claim 1, wherein the treatment comprises at least two of steps (a), (b) and (c).

3. The process according to claim 1, wherein the protic compound is selected from the group consisting of water, an aqueous salt solution, a dilute inorganic acid, a monohydric or polyhydric aliphatic or aromatic alcohol, a monovalent or polyvalent aliphatic or aromatic carboxylic acid and a monovalent or polyvalent aliphatic or aromatic amine.

4. The process according to claim 1, wherein protic compound is water or an aqueous salt solution.

5. The process according to claim 1, wherein the gas is selected from the group consisting of a noble gas, nitrogen, carbon dioxide, dinitrogen monoxide, water vapor, a hydrocarbon and a fluorochlorohydrocarbon.

6. The process according to claim 1, wherein the gas is selected from the group consisting of carbon monoxide and hydrogen.

7. The process according to claim 1, wherein the reacting in the presence of a catalyst and the treatment are carried out in apparatuses which are different from one another.

8. The process according to claim 1, wherein the separating of the solid metallic catalyst constituents is by centrifugation.

9. The process according to claim 1, wherein the separating of the solid metallic catalyst constituents is by a self-desludging disk separator.

10. The process according to claim 1, wherein the separating of the solid metallic catalyst constituents is by filtration.
